(19) United States
(12) Plant Patent Application Publication
(10) Pub. No.: US 2006/0075532 P1
(43) Pub. Date: Apr. 6, 2006

(54) Dianella revoluta plant named 'DRG04'

(76) Inventor: Todd Anthony Layt, New South Wales (AU)

Correspondence Address: MYERS BIGEL SIBLEY & SAJOVEC, PO BOX 37428, RALEIGH, NC, 27627, US

(21) Appl. No.: 11/239,545

(22) Filed: Sep. 29, 2005

Related U.S. Application Data
(60) Provisional application No. 60/614,795, filed on Sep. 30, 2004.

(51) Int. Cl. A01H 5/00

(52) U.S. Cl. PLT/263

(57) ABSTRACT

‘DRG04’ is a distinctive variety of Dianella revoluta, which is characterized by the combination of a dense compact clumping habit with red basal shoots and a slight weeping of clumps with age. ‘DRG04’ is further characterized by its glaucous leaves, which result in a two-toned leaf colour when viewed from a distance, with a glossy green upper side leaf colour and a dull green (slightly blue-toned) lower side leaf colour. When observed up-close the leaves are a yellow-green. ‘DRG04’ is also distinguished by the ability to be easily and productively propagated by division.

### RELATED APPLICATION INFORMATION

This application claims the benefit of U.S. Provisional Application Ser. No. 60/614,795, filed Sep. 30, 2004, the disclosure of which is incorporated herein by reference in its entirety.

### LATIN NAME OF THE GENUS AND SPECIES

The Latin name of the novel variety disclosed herein is Dianella revoluta.

### VARIETY DENOMINATION

The inventive variety of Dianella revoluta disclosed herein has been given the varietal denomination ‘DRG04’.

### BACKGROUND OF THE INVENTION

The present invention relates to a new and distinct perennial Spreading Flax Lilly variety of Dianella revoluta, which has been given the varietal denomination of ‘DRG04’. Its market class is that of an ornamental grass-like plant. ‘DRG04’ is intended for use in landscaping and as a decorative grass-like plant.

An application for plant breeders' rights for variety ‘DRG04’ has been filed with the Australian Plant Breeders' Rights Office, and was first gazetted in the Plant Varieties Journal in October 2003 under Application No. 2003/289.
Parentage:

The Dianella revoluta variety ‘DRG04’ was first discovered in October 1996 in an Australian nursery in the state of New South Wales after an extensive breeding program. The parent of ‘DRG04’, ‘Queanbeyan Ecotype’ (‘DR4000’, unpatented), is characterized by tall plant height, broad basal shoot width, weaker plant density, medium leaf size, dull green leaf colour and frequent leaf spotting.

In 1995, Dianella species were grown together in an open bed covering approximately 250 square metres of area. They were grown in groups of species and ecotypes including Dianella revoluta collected from the Queanbeyan area, and other regions. The other Dianella species present were longifolia, tasmanica and caerulea. The plants were open pollinated with possible assisted pollination from general shaking of flower stems onto each other.

Seeds were collected and sown from the ‘Queanbeyan Ecotype’ plants in December 1995. Approximately 10,000 plants were grown. In October 1996, using the selection criteria of contrasting upper and lower side leaf colour and dense growth habit, a single plant was identified as having a glossy green upper side leaf colour and dull green lower side leaf colour. This plant was selected, potted into a 140 mm pot for further evaluation, and named ‘DRG04’.
Asexual reproduction: ‘DRG04’ was first asexually propagated by division in April 1997 in the state of New South Wales, Australia. ‘DRG04’ was asexually reproduced again during November 1997 and April 1998 and confirmed to be stable in character. The distinctive characteristics of the inventive variety, ‘DRG04’, have remained stable and true to type from generation to generation through successive cycles of asexual reproduction including vegetative division and micropropagation.

### SUMMARY OF THE INVENTION

‘DRG04’ is a distinctive variety of Dianella revoluta, which is characterized by the combination of a dense compact clumping habit with red basal shoots and a slight weeping of clumps with age. ‘DRG04’ is further characterized by its glaucous leaves, which result in a two-toned leaf colour when viewed from a distance, with a glossy green upper side leaf colour and a dull green (slightly blue-toned) lower side leaf colour. When observed up-close the leaves are a yellow-green. ‘DRG04’ is also distinguished by the ability to be easily and productively propagated by division.

### BRIEF DESCRIPTION OF THE FIGURES

The photographs in the drawings were made using conventional techniques and show the colours as true as reasonably possible by conventional photography. Colours in the photographs may differ slightly from the colour values cited in the detailed botanical description, which accurately describe the colours of the new Dianella revoluta.

FIG. 1 shows two ‘DRG04’ plants and illustrates the red basal shoots, two-toned leaf colour, and dense compact growth habit that are characteristic of the variety.

FIG. 2 shows leaf width and glaucosity of ‘DRG04’ compared with other Dianella revoluta varieties, ‘DR5000’ (U.S. Plant patent application Ser. No. 10/883,942, filed Jul. 2, 2004) and ‘DR4000’ (‘Queanbeyan ecotype’, unpatented).

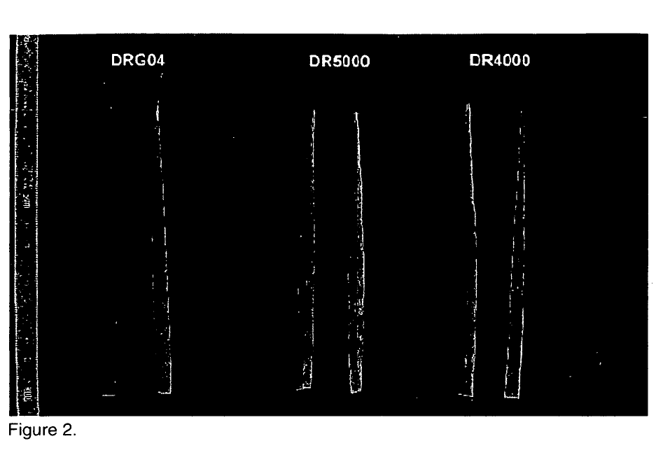

### BOTANICAL DESCRIPTION OF THE PLANT

The following is a detailed botanical description of a new and distinct variety of a Dianella revoluta ornamental grass-like plant known as ‘DRG04’. The descriptions disclosed herein are based upon observations of the plant grown in 140 mm nursery pots and field plots in New South Wales, Australia. The plants were approximately 12-months-old at the time of observation. Except as indicated, all colors cited herein refer to The Royal Horticultural Society Colour Chart (The Royal Horticultural Society, London, 2001 edition).

Those skilled in the art will appreciate that certain characteristics will vary with older or, conversely, younger plants. ‘DRG04’ has not been observed under all possible environmental conditions. Where dimensions, sizes, colors and other characteristics are given, it is to be understood that such characteristics are approximations or averages set forth as accurately as practicable. The phenotype of the variety may differ with variations in the environment such as season, temperature, light intensity, day length, cultural conditions and the like.

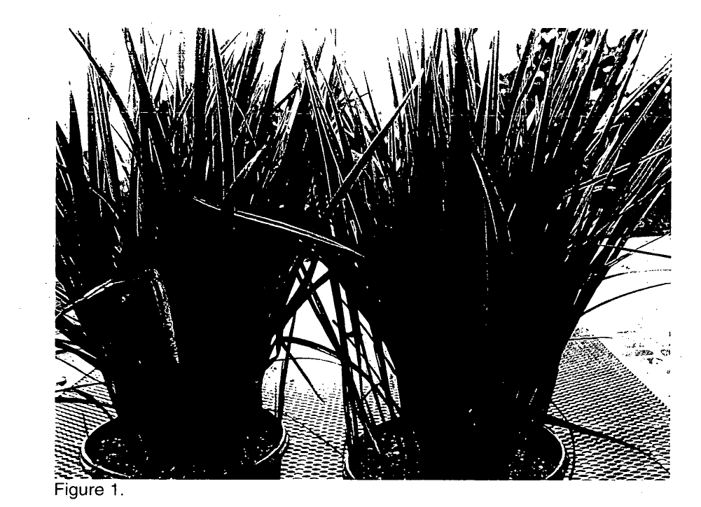

‘DRG04’ is a perennial Dianella revoluta plant, which was produced through an innovative breeding program. After its selection, ‘DRG04’ was asexually propagated by division and micropropagation. ‘DRG04’ has a dense compact clumping form with red basal shoots, and a slight weeping of clumps with age. The leaves of ‘DRG04’ are glaucous and as a result have a two-toned leaf colour when viewed from a distance, with a glossy green upper side leaf colour and dull (slightly blue-toned) green lower side leaf colour. When observed up-close the leaves appear yellow-green. A botanical description of ‘DRG04’ and a comparison with other varieties of Dianella revoluta are provided below. Representative Dianella revoluta ‘DRG04’ plants are shown in FIG. 1.
Technical description of the variety:
   Growth habit.—‘DRG04’ forms a dense compact clumping tussock. Its growth habit is erect, its height is medium-tall (mean 53.3 cm), and the density of its shoots is strong.
   Foliage.—Leaf attitude erect, width medium (mean 9.0 mm), upper side colour (waxiness removed) is yellow-green (RHS 146A), lower side colour (waxiness removed) is yellow-green (RHS 147B), upper side glaucosity medium, shape ligulate, apex acute, cross section concave, margin flat to weakly revolute.
   Basal shoots.—Red.
   Basal sheath.—Anthocyanin coloration red-brown to purple, intensity of colour strong.
   Environmental tolerances.—‘DRG04’ has shown potential for shade tolerance. The winter hardiness of ‘DRG04’ is at least to -12 degrees Celsius. ‘DRG04’ has good winter color retention.
   Disease resistance and susceptibility.—‘DRG04’ has excellent tolerance to pests and diseases.

These features and other characteristics of the plant are apparent from the figures.
‘DRG04’ compared to other varieties of Dianella revoluta:

Grouping characteristics used to identify the most similar varieties of common knowledge to ‘DRG04’ were medium-tall plant height and dense growth habit. Based on this the ‘Queanbeyan Ecotype’ (‘DR4000’) was selected as the most similar suitable comparator. ‘DR5000’, a sibling cultivar, was also included in the trial. No other similar varieties were identified.

The comparative trial of Dianella revoluta ‘DRG04’ with other D. revoluta was conducted in open beds in Summer 2002 through Autumn 2003 in Clarendon, New South Wales, Australia. The plants for this trial were propagated from divisions and planted into 130 mm pots filled with soilless potting mix. Nutrition was maintained with slow release fertilizers and pest and disease treatments were applied as required. The plants did not flower during the trial. Trial design included fifteen pots of each variety arranged in a completely randomized design. Measurements were taken from ten plants at random with one sample taken per plant.

In comparing ‘DRG04’ with the ‘Queanbeyan Ecotype’ and ‘DR5000’, ‘DRG04’ has an upper side leaf colour of glossy green (making the leaf two-toned as lower side is dull), whereas the ‘Queanbeyan Ecotype’ (seed parent of ‘DRG04’) has a dull green upper leaf colour and ‘DR5000’ has a blue-green upper leaf colour. ‘DRG04’ has a dense growth habit with many shoots and a somewhat weeping habit at maturity, whereas ‘DR5000’ has a dense upright growth habit and does not weep at maturity, and the ‘Queanbeyan Ecotype’ is less dense and more upright. ‘DRG04’ has a medium width basal shoot, whereas ‘DR5000’ has a narrow-medium basal shoot width, and the ‘Queanbeyan Ecotype’ has a broad basal shoot width. ‘DRG04’ has an improved resistance to spotting compared to other varieties. ‘DRG04’ divides for nursery production better than any other known Dianella revoluta, producing both more divisions and at a higher success rate. ‘DRG04’ is more compact in height compared to most other Dianella revoluta, with the exception of ‘DR5000’. Further comparisons are presented in Table I.

TABLE I
Comparison of Dianella revoluta varieties

| | | |
|---|---|---|
| | | ‘Queanbeyan |
| | | Ecotype’ |
| ‘DRG04’ | ‘DR5000’ | (‘DR4000’) |

PLANT: HEIGHT (cm) -

| | | | |
|---|---|---|---|
| Mean | 53.3 | 24.7 | 57.1 |
| Std deviation | 2.4 | 1.9 | 4.9 |
| LSD/sig | 3.82 | P ≦ 0.01 | ns |

PLANT: DENSITY OF SHOOTS

| | | |
|---|---|---|
| strong | strong | weak |

LEAF: WIDTH (mm)

| | | | |
|---|---|---|---|
| Mean | 9.0 | 9.1 | 10.4 |
| Std deviation | 1.0 | 1.0 | 1.0 |
| LSD/sig | 1.13 | ns | P ≦ 0.01 |

LEAF: GLAUCOSITY

| | | |
|---|---|---|
| medium | strong | medium |

LEAF: COLOUR (RHS 2001, where denoted * better match
with 1995 chart)

| | | | |
|---|---|---|---|
| Upper side | 146A | 147A | 146A |
| Lower side | 147B | 189A* (1995) | 146C-D |

LEAF: CROSS SECTION

| | | |
|---|---|---|
| concave | concave | flat-revolute |

BASAL SHEATH INTENSITY OF COLOUR

| | | |
|---|---|---|
| strong | medium | medium |

That which is claimed is:

1. A new and distinct variety of Dianella revoluta plant named ‘DRG04’, substantially as described and illustrated herein.
